(19) United States
(12) Patent Application Publication
(10) Pub. No.: US 2003/0078272 A1
(43) Pub. Date: Apr. 24, 2003

(54) Methods of use of fluoroquinolone compounds against ciprofloxacin-resistant and ciprofloxacin-sensitive pathogenic bacteria

(75) Inventors: Peter C. Appelbaum, Hershey, PA (US); Lois M. Ednie, Hershey, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/208,082

(22) Filed: Jul. 29, 2002

Related U.S. Application Data
(63) Continuation of application No. 09/611,998, filed on Jul. 7, 2000, now abandoned.
(60) Provisional application No. 60/142,724, filed on Jul. 8, 1999.

(51) Int. Cl. A61K 31/496; A61K 31/4709

(52) U.S. Cl. 514/253.08; 514/312

(57) ABSTRACT

This invention relates, in part, to newly identified methods of using quinolone antibiotics, particularly a gemifloxacin compound against certain pathogenic bacteria.

### CROSS-REFERENCE TO RELATED APPLICATION

This application claims benefit to the earlier provisional U.S. application Serial No. 60/142,724, filed Jul. 8, 1999, the contents of which are incorporated herein by reference in their entirety.

### FIELD OF THE INVENTION

This invention relates, in part, to newly identified methods of using quinolone antibiotics, particularly a gemifloxacin compound against ciprofloxacin-sensitive and ciprofloxacin-resistant bacteria, such as pneumococci, especially Streptococcus pneumoniae.

### BACKGROUND OF THE INVENTION

Quinolones have been shown to be effective to varying degrees against a range of bacterial pathogens. However, as diseases caused by these pathogens are on the rise, there exists a need for antimicrobial compounds that are more potent than the present group of quinolones.

Gemifloxacin mesylate (SB-265805) is a novel fluoroquinolone useful as a potent antibacterial agent. Gemifloxacin compounds are described in detail in patent application PCT/KR98/00051 published as WO 98/42705. Patent application EP 688772 discloses novel quinolone(naphthyridine)carboxylic acid derivatives, including anhydrous (R,S)-7-(3-aminomethyl-4-methoxyiminopyrrolidin-1-yl)-1-cyclopropyl-6-fluoro-4-oxo-1,4-dihydro-1,8-naphthyridine-3-carboxylic acid of formula I.

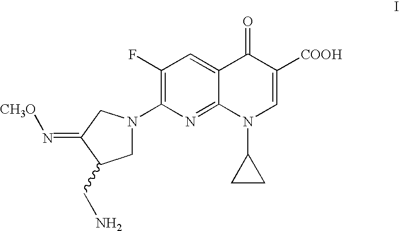

PCT/KR98/00051 discloses (R,S)-7-(3-aminomethyl-4-syn-methoxyimino-pyrrolidin-1-yl)-1-cyclopropyl-6-fluoro-4-oxo-1,4-dihydro-1,8-naphthyridine-3-carboxylic acid methanesulfonate and hydrates thereof including the sesquihydrate.

Provided herein is a significant discovery made using a gemifloxacin compound against ciprofloxacin-sensitive and ciprofloxacin-resistant pneumococci, demonstrating the activity of the gemifloxacin compound used was superior to a number of quinolones as described in more detail herein. Gemifloxacin compounds are valuable compounds for the treatment of infections caused by a range of bacterial pathogens, including those resistant to usual oral therapy, thereby filling an unmet medical need.

The incidence of pneumococci resistant to penicillin G and other β-lactam and non-β-lactam compounds has increased worldwide at an alarming rate, including in the USA Major foci of resistance currently include South Africa, Spain, Central and Eastern Europe, and parts of Asia (P. C. Appelbaum, Clin. Infect. Dis. 15:77-83, 1992). In the USA there has been an increase in resistance to penicillin from <5% before 1989 (including <0.02% of isolates with MICs≧2.0 μg/ml) to 6.6% in 1991-1992 (with 1.3% of isolates with MICs≧2.0 μg/ml), (Breiman, et al., JAMA 271:1831-1835, 1994) and to 23.6% (360) of 1527 strains during 1994-1995 (Doern, et al., Antimicrob. Agents Chemother. 40:1208-1213, 1996). It is also important to note the high rates of isolation of penicillin-intermediate and -resistant pneumococci (approximately 30%) in middle ear fluids from patients with refractory otitis media, compared to other isolation sites (Block, et al., Pediatr. Infect. Dis. 14:751-759, 1995).

There is an urgent need of oral compounds for out-patient treatment of otitis media and other respiratory tract infections caused by penicillin-intermediate and -resistant pneumococci (Friedland, et al., N. Eng. J. Med., 331:377-382, 1994 and M. R. Jacobs, Clin. Infect. Dis. 15:119-127, 1992). Older quinolones such as ciprofloxacin and ofloxacin yield moderate in vitro activity against pneumococci, with MICs clustering around the breakpoints (Pankuch, et al., J. Antimicrob. Chemother. 35:230-232, 1995). Methods for routine susceptibility testing of pneumococci include broth microdilution and disk diffusion (the methods recommended by the NCCLS, agar dilution and E-test (Methods for Dilution Antimicrobial Susceptibility Tests for Bacteria that Grow Aerobically, 4th Edition, NCCLS. Villanova, Pa., 1997 and Performance Standards for Antimicrobial Disk Susceptibility Tests, 6th Edition, NCCLS, Villanova, Pa., 1997). NCCLS recommends incubation of microdilution MICs in ambient air, but disk diffusion in CO₂, (Methods for Dilution Antimicrobial Susceptibility Tests for Bacteria that Grow Aerobically, 4th Edition, NCCLS, Villanova, Pa., 1997 and Performance Standards for Antimicrobial Disk Susceptibility Texts, 6th Edition, NCCLS, Villanova, Pa., 1997) while the manufacturer of the E-test recommends incubation in CO₂. There is no standard recommendation for agar dilution pneumococcal MIC testing methodology, although the method has been extensively used in the art (M. R. Jacobs, Clin. Infect. Dis. 15:119-127, 1992; Pankuch et al., J. Antimicrob. Chemother. 35:230-232, 1995; and Clark, et al., J. Clin. Microbiol. 36:3579-3584, 1998).

If new compounds are to be tested for anti-pneumococcal activity in the clinical laboratory, methodology must be standardized. The current study used microdilution and agar dilution (air), E-test (air and CO₂) and disk diffusion (air and CO₂) to test activity of gemifloxacin (SB-265805; LB 20304a), a new fluoronaphthyridone with a novel pyrrolidone substituent, with good Gram positive and negative activity, (Oh, et al., Antimicrob. Agents Chemother. 40:1564-1568, 1996; Cormican, et al., Antimicrob. Agents Chemother. 41:204-211, 1997; Hohl, et al., Clin. Microbiol. Infect. 4:280-984, 1998; Kelly, et al., Program and Abstracts of the Thirty-Eighth Interscience Conference on Antimicrobial Agents and Chemotherapy, San Diego, Calif., USA 1998. American Society for Microbiology: Washington, D.C. 1998, pages 254, Abstract F-87) against 200 pneumococci, including those with raised penicillin and quinolone MICs.

### SUMMARY OF THE INVENTION

An object of the invention is a method for modulating metabolism of ciprofloxacin-resistant and/or ciprofloxacin-sensitive bacteria comprising the step of contacting ciprofloxacin-resistant and/or ciprofloxacin-sensitive bacteria with an antibacterially effective amount of a composition comprising a quinolone, particularly a gemifloxacin compound, or an antibacterially effective derivative thereof.

A further object of the invention is a method wherein said ciprofloxacin-resistant and/or ciprofloxacin-sensitive bacteria is selected from the group consisting of: ciprofloxacin-susceptible pneumococci having an MIC≦4 μg/ml of ciprofloxacin; ciprofloxacin-resistant pneumococci having an MIC≧8 μg/ml of ciprofloxacin; ciprofloxacin-susceptible Streptococcus pneumoniae having an MIC≦4 μg/ml of ciprofloxacin; and ciprofloxacin-resistant Streptococcus pneumoniae having an MIC≧8 μg/ml of ciprofloxacin.

Also provided by the invention is a method of treating or preventing a bacterial infection by ciprofloxacin-resistant and/or ciprofloxacin-sensitive bacteria comprising the step of administering an antibacterially effective amount of a composition comprising a quinolone, particularly a gemifloxacin compound to a mammal suspected of having or being at risk of having an infection with ciprofloxacin-resistant and/or ciprofloxacin-sensitive bacteria.

A preferred method is provided wherein said modulating metabolism is inhibiting growth of said bacteria or killing said bacteria.

A further preferred method is provided wherein said contacting said bacteria comprises the further step of introducing said composition into a mammal, particularly a human.

Further preferred methods are provided by the invention wherein said bacteria is selected from the group consisting of: ciprofloxacin-susceptible pneumococci having an MIC≦4 μg/ml of ciprofloxacin; ciprofloxacin-resistant pneumococci having an MIC≧8 μg/ml of ciprofloxacin; ciprofloxacin-susceptible Streptococcus pneumoniae having an MIC≦4 μg/ml of ciprofloxacin; and ciprofloxacin-resistant Streptococcus pneumoniae having an MIC≧8 μg/ml of ciprofloxacin.

Various changes and modifications within the spirit and scope of the disclosed invention will become readily apparent to those skilled in the art from reading the following descriptions and from reading the other parts of the present disclosure.

### BRIEF DESCRIPTION OF THE DRAWINGS

FIG. 1 shows a graphical depiction of agar dilution MICs obtained with ciprofloxacin compared with those obtained with gemifloxacin against 161 ciprofloxacin-susceptible 39 and ciprofloxacin-resistant strains

FIG. 2 shows a graphical depiction of gemifloxacin zone diameters (mm) in CO₂ compared with microdilution MICs in air for all 200 strains

### DESCRIPTION OF THE INVENTION

The present invention provides, among other things, methods for using a composition comprising a quinolone, particularly a gemifloxacin compound against ciprofloxacin-susceptible pneumococci having an MIC≦4 μg/ml of ciprofloxacin; ciprofloxacin-resistant pneumococci having an MIC≧8 μg/ml of ciprofloxacin; ciprofloxacin-susceptible Streptococcus pneumoniae having an MIC≦4 μg/ml of ciprofloxacin; and ciprofloxacin-resistant Streptococcus pneumoniae having an MIC≧8 μg/ml of ciprofloxacin.

As used herein “gemifloxacin compound(s)” means a compound having antibacterial activity described in patent application PCT/KR98/00051 published as WO 98/42705, or patent application EP 688772.

This invention was based, in part, on analyses evaluating the comparative activity of gemifloxacin against various bacterial pathogens.

The invention was based, in part, on experiments using agar dilution, microdilution (both in air), E-test and disk diffusion (both in air and CO₂) were used to test activity of gemifloxacin (SB-265805) against 161 ciprofloxacin-susceptible (cipro-S) (MIC≦4 μg/ml) and 39 ciprofloxacin-resistant (cipro-R) (MIC≧8 μg/ml) pneumococci. By agar, gemifloxacin MIC₅₀S/MICs₉₀S (μg/ml) for cipro-S and -R strains were 0.03/0.03 and 0.25/0.5, respectively. Results of agar dilution MICs for ciprofloxacin vs gemifloxacin for all 200 strains showed a linear correlation. Comparing the three MIC methods for all strains, MICs (μg/ml) were practically identical: agar dilution, range 0.004-1, MIC₅₀ 0.03, MIC₉₀ 0.25; Microdilution, range 0.004-0.5, MIC₅₀ 0.016, MIC₉₀ 0.125; E-test (air), range 0.008-0.5, MIC₅₀ 0.016, MIC₉₀ 0.125; E-test (CO₂), range 0.00-0.5, MIC₅₀ 0.016, MIC₉₀ 0.25. With agar dilution as the standard, 187/200 strains (93.5%) gave essential agreement (±1 log₂ dilution) with microdilution, and 196/200 (98%) with the E-test (both in air and CO₂). Incubation of E-tests in air and CO₂ gave identical results. With a 0.5 μg/ml breakpoint, no major or very major errors occurred. When 5 μg gemifloxacin disks were incubated in CO₂, all cipro-S strains yielded gemifloxacin diameters ≧26 mm. Gemifloxacin diameters in CO₂ for cipro-R strains varied between 18-31 but were mostly 21-26 mm. Using a gemifloxacin breakpoint of 0.5 μg/ml, diameters in CO₂ of ≧20 mm for S and ≦19 mm for R strains are proposed: all strains but one (with an agar dilution MIC 1 μg/ml) had gemifloxacin MICs of ≦0.5 μg/ml, and all but one had zone diameters in CO₂>20 mm. Most diameters in air were 1-3 mm wider than in CO₂, but S and R results were identical. Results show that i) Gemifloxacin is very active against cipro-S and -R strains; ii) susceptibility to gemifloxacin can be reliably tested by agar and microdilution, E-test and disk diffusion; iii) CO₂ does not significantly affect gemifloxacin pneumococcal susceptibility results.

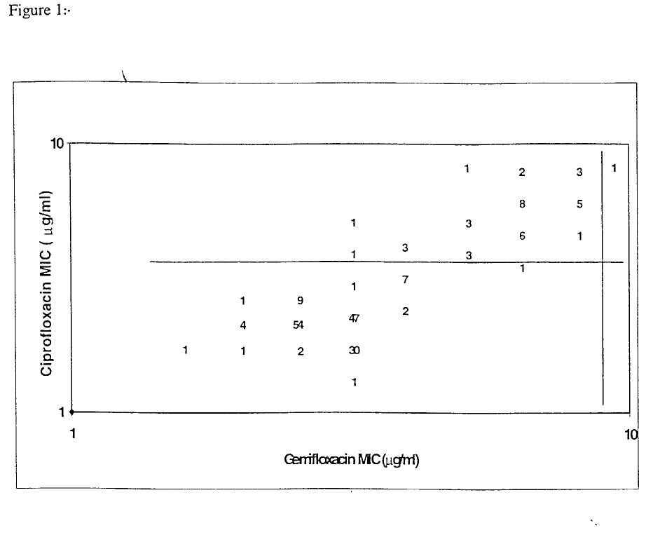

Results of MIC testing of gemifloxacin with the four methods used are presented in Table 1, and results (agar dilution MIC) broken down by ciprofloxacin susceptibility in Table 2 By agar dilution, ciprofloxacin MICs (μg/ml) for all strains ranged between 0.5-≧64, with an MIC₅₀ of 2 and an MIC₉₀ of 16. By contrast, gemifloxacin MICs, which were practically identical with all methods, ranged between 0.004-1.0 μg/ml, with MIC₅₀s between 0.016-0.03 μg/ml and MIC₉₀s between 0.125-0.25 μg/ml with agar dilution and microdilution in air, and E-test (both in air and CO₂). Incubation of E-tests in CO₂ did not significantly influence MICs When strains with ciprofloxacin MICs≦4.0 μg/ml) were separated from strains with ciprofloxacin MICs≧8 μg/ml, gemifloxacin MIC₅₀/₉₀ values (μg/ml) by agar dilution were 0.03/0.03 and 0.25/0.5, respectively. By contrast, ciprofloxacin MIC₅₀/₉₀ values (μg/ml) for susceptible and resistant strains were 1/2 and 32/≧64, respectively (Table 2) Results of agar dilution MICs for ciprofloxacin vs gemifloxacin for all 200 strains tested showed a linear correlation (FIG. 1).

Agreements of microdilution and E-test (air and CO₂) with agar dilution (used as the reference method) and E-tests in air versus CO₂ are presented in Table 3. As can be seen, 187/200 strains (93.5%) gave essential agreement (±log₂ dilution) with microdilution, and 98.0% with the E-test (both in air and CO₂). With a preliminary breakpoint of 0.5 μg/ml, no major or very major discrepancies were found with microdilution in air or E-test in air or CO₂. E-tests incubated in air gave virtually identical results to those in CO₂ (Table 3).

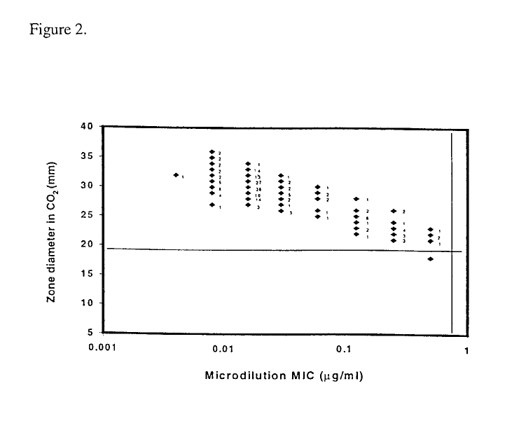

With disks incubated in CO₂, all quinolone-susceptible strains yielded zone diameters ≧26 mm; values in air were ≧28 mm. Zone diameters for quinolone-resistant strains in CO₂ varied between 18 and 31 mm but were mostly 21-26 mm; zone diameters in air were a few mm wider, but were also mostly <31 mm. Correlation between microdilution in air, the method recommended by NCCLS, and disk diffusion (incubated in CO₂) is presented in FIG. 2. Using a gemifloxacin breakpoint of 0.5 μg/ml, ≧20 mm for susceptible and ≦19 mm (resistant) are proposed, as all strains but one (with agar dilution) had MICs of ≦0.5 μg/ml, and all but one strain had zones >20 mm. With a breakpoint of 0.25 μg/ml, zone diameters (mm) of ≧23 (susceptible), 21-22 (intermediate) and ≦20 (resistant) are indicated. Zone diameters in air were usually 1-3 mm wider than those in CO₂.

MICs of gemifloxacin against pneumococci are similar to those described previously, including MICs (μg/ml) of 0.004-0.06 if ciprofloxacin MICs are ≦4 (Oh, et al., Antimicrob. Agents Chemother., 40:1564-1568, 1996; Cormican, et al., Antimicrob. Agents Chemother., 41:204-211, 1997; Hohl, et al., Clin. Microbiol. Infect. 4:280-284, 1998; and Kelly, et al., Program and Abstracts of the Thirty-Eighth Interscience Conference on Microbial Agents and Chemotherapy, San Diego, Calif., USA 1998. American Society for Microbiology: Washington, D.C., 1998, page 254, Abstract F-87) and 0.03-1.0 if ciprofloxacin MICs are 8.0-64.0. Additionally, MICs did not differ significantly with agar and microdilution incubated in air, and E-tests incubated in air and CO₂. Other studies demonstrated the same findings with levofloxacin (Clark, et al., J. Clin. Microbiol. 36:3579-3584, 1998). Disk diffusion testing showed zone sizes slightly smaller in CO₂ than in air. Determination of breakpoints, both by disk diffusion and MIC, must await further studies, but ciprofloxacin-resistant strains with gemifloxacin MICs of 0.06-1.0 μg/m by agar dilution gave zone diameters In CO₂ between 21 and 31 mm (with the exception of one strain with a zone diameter of 18 mm), and most quinolone-susceptible strains yielded zone diameters >25 mm.

Results of this study indicate an excellent correlation between agar dilution, microdilution and E-test methods, and all methods can confidently be recommended for pneumococcal susceptibility testing with gemifloxacin. Using a gemifloxacin breakpoint of 0.5 μg/ml, ≧20 mm for susceptible and ≦19 mm are proposed (see Results and FIG. 2). With a breakpoint of 0.25 μg/ml, zone diameters (mm) of ≧23 (susceptible), 21-22 (intermediate) and ≦20 (resistant) are indicated.

TABLE 1
Gemifloxacin MICs (μg/ml) with the three
methods tested against 200 strains

| Method | MIC range | MIC₅₀ | MIC₉₀ |
|---|---|---|---|
| Agar dilution (air) | 0.004-1.0 | 0.03 | 0.25 |
| Microdilution (air) | 0.004-0.5 | 0.016 | 0.125 |
| E-test (air) | 0.008-0.5 | 0.016 | 0.125 |
| E-test (CO₂) | 0.008-0.5 | 0.016 | 0.25 |

TABLE 2
Comparison of agar dilution MIC results for ciprofloxacin-susceptible (161) and
resistant (39) strains

| | |
|---|---|
| Ciprofloxacin MIC ≦4.0 (μg/ml) | Ciprofloxacin MIC ≧8.0 (μg/ml) |

| Antimicrobial | Range | MIC₅₀ | MIC₉₀ | Range | MIC₅₀ | MIC₉₀ |
|---|---|---|---|---|---|---|
| Gemifloxacin | 0.004-0.06 | 0.03 | 0.03 | 0.03-1 | 0.25 | 0.5 |
| Ciprofloxacin | 0.5-4 | 1 | 2 | 8-≧64 | 32 | ≧64 |

TABLE 3
Results of gemifloxacin pneumoccocal susceptibility testing by four methods using agar
dilution as the reference method

| | Number of strains with log₂ ratios of reference to | | | | | | | | |
|---|---|---|---|---|---|---|---|---|---|
| Method | test MICs of method A vs method B | | | | | | | | Number ±1 log₂ dilution |
| Method A | Method B | ≧+3 | +2 | +1 | 0 | −1 | −2 | −3 | |
| Agar dilution | Microdilution | 0 | 0 | 5 | 83 | 99 | 13 | 0 | 187 |
| Agar dilution | E-test | 0 | 0 | 7 | 87 | 102 | 4 | 0 | 196 |
| | (air) | | | | | | | | |
| Agar dilution | E-test (CO₂) | 0 | 0 | 12 | 95 | 89 | 4 | 0 | 196 |
| E-test (air) | E-test (CO₂) | 0 | 0 | 27 | 164 | 9 | 0 | 0 | 200 |

The invention provides a method for modulating metabolism of ciprofloxacin-resistant and/or ciprofloxacin-sensitive bacteria. Skilled artisans can readily choose ciprofloxacin-resistant and/or ciprofloxacin-sensitive bacteria or patients infected with or suspected to be infected with these organisms to practice the methods of the invention. Alternatively, the bacteria useful in the methods of the invention may be those described herein.

The contacting step in any of the methods of the invention may be performed in many ways that will be readily apparent to the skilled artisan. However, it is preferred that the contacting step is a provision of a composition comprising a gemifloxacin compound to a human patient in need of such composition or directly to bacteria in culture medium or buffer.

For example, when contacting a human patient or contacting said bacteria in a human patient or ii vitro, the compositions comprising a quinolone, particularly a gemifloxacin compound, preferably pharmaceutical compositions may be administered in any effective, convenient manner including, for instance, administration by topical, oral, anal, vaginal, intravenous, intraperitoneal, intramuscular, subcutaneous, intranasal or intradermal routes among others.

It is also preferred that these compositions be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a compound of the invention, a quinolone, preferably a gemifloxacin compound, and a pharmaceutically acceptable carrier or excipient. Such carriers may include, but are not limited to, saline, buffered saline, dextrose, water, glycerol, ethanol and combinations thereof. The formulation should suit the mode of administration.

Quinolone compounds, particularly gemifloxacin compounds and compositions of the methods of the invention may be employed alone or in conjunction with other compounds, such as bacterial efflux pump inhibitor compounds or antibiotic compounds, particularly non-quinolone compounds, e.g., beta-lactam antibiotic compounds.

In therapy or as a prophylactic, the active agent of a method of the invention is preferably administered to an individual as an injectable composition, for example as a sterile aqueous dispersion, preferably an isotonic one.

Alternatively, the gemifloxacin compounds or compositions in the methods of the invention may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the antibacterially effective amount is a daily dosage level of the active agent from 0.001 mg/kg to 10 mg/kg, typically around 0.1 mg/kg to 1 mg/kg, preferably about 1 mg/kg. A physician, in any event, will determine an actual dosage that is most suitable for an individual and will vary with the age, weight and response of the particular individual. The above dosages are exemplary of the average case. There can, of course, be individual instances where higher or lower dosage ranges are merited, and such are within the scope of this invention. It is preferred that the dosage is selected to modulate metabolism of the bacteria in such a way as to inhibit or stop growth of said bacteria or by killing said bacteria. The skilled artisan may identify this amount as provided herein as well as using other methods known in the art, e.g. by the application MIC tests.

A further embodiment of the invention provides for the contacting step of the methods to further comprise contacting an in-dwelling device in a patient. In-dwelling devices include, but are not limited to, surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters.

A quinolone, particularly a gemifloxacin compound or composition of the invention may be administered by injection to achieve a systemic effect against relevant bacteria, preferably a ciprofloxacin-resistant and/or ciprofloxacin-sensitive bacteria, shortly before insertion of an in-dwelling device. Treatment may be continued after surgery during the in-body time of the device. In addition, the composition could also be used to broaden perioperative cover for any surgical technique to prevent bacterial wound infections caused by or related to ciprofloxacin-resistant and/or ciprofloxacin-sensitive bacteria.

In addition to the therapy described above, a gemifloxacin compound or composition used in the methods of this invention may be used generally as a wound treatment agent to prevent adhesion of bacteria to matrix proteins, particularly ciprofloxacin-resistant and/or ciprofloxacin-sensitive bacteria, exposed in wound tissue and for prophylactic use in dental treatment as an alternative to, or in conjunction with, antibiotic prophylaxis.

Alternatively, a quinolone, particularly a gemifloxacin compound or composition of the invention may be used to bathe an indwelling device immediately before insertion. The active agent will preferably be present at a concentration of 1 μg/ml to 10 mg/ml for bathing of wounds or indwelling devices.

Also provided by the invention is a method of treating or preventing a bacterial infection by ciprofloxacin-resistant and/or ciprofloxacin-sensitive bacteria comprising the step of administering an antibacterially effective amount of a composition comprising a quinolone, particularly a gemifloxacin compound to a mammal, preferably a human, suspected of having or being at risk of having an infection with ciprofloxacin-resistant and/or ciprofloxacin-sensitive bacteria.

While a preferred object of the invention provides a method wherein said ciprofloxacin-resistant and/or ciprofloxacin-sensitive bacteria is selected from the group consisting of: ciprofloxacin-susceptible pneumococci having an MIC≦4 μg/ml of ciprofloxacin;

ciprofloxacin-resistant pneumococci having an MIC≧8 μg/ml of ciprofloxacin;

ciprofloxacin-susceptible Streptococcus pneumoniae having an MIC≦4 μg/ml of ciprofloxacin; and

ciprofloxacin-resistant Streptococcus pneumoniae having an MIC≧8 μg/ml of ciprofloxacin.

Other ciprofloxacin-resistant and/or ciprofloxacin-sensitive bacteria may also be included in the methods. The skilled artisan may identify these organisms as provided herein as well as using other methods known in the art, e.g. MIC tests.

Preferred embodiments of the invention include, among other things, methods wherein said composition comprises gemifloxacin, or a pharmaceutically acceptable derivative thereof.

### EXAMPLES

The present invention is further described by the following examples. The examples are provided solely to illustrate the invention by reference to specific embodiments. This exemplification's, while illustrating certain specific aspects of the invention, do not portray the limitations or circumscribe the scope of the disclosed invention.

All examples were carried out using standard techniques, which are well known and routine to those of skill in the art, except where other vise described in detail.

All parts or amounts set out in the following examples are by weight, unless otherwise specified.

### Example 1

### Isolates and Antimicrobial Agents

Of 200 recently clinically isolated strains of pneumococci, 68 were penicillin-susceptible (MIC<1.0 μg/ml); 67 were penicillin intermediate (MIC 0.1-1.0 μg/ml) and 65 penicillin resistant (MIC≧2.0 μg/ml). The 200 strains included 39 with raised quinolone MICs (ciprofloxacin MICs≧8 μg/ml-21 penicillin susceptible, 12 intermediate, 6 penicillin resistant). Cultures were maintained at −70° C. in double-strength skim milk (Difco Laboratories, Detroit, Mich.). Gemifloxacin susceptibility powder, disks and E-tests (AB Biodisk, Solna, Sweden) were obtained from SmithKline Beecham Laboratories, Collegeville, Pa., USA.

### Example 2

### Agar Dilution MICs

These were performed (Jacobs, et al., Clin. Infect. Dis. 15:119-127, 1992 and Clark, et al., J. Clin. Microbiol. 36:3579-3584, 1998) on Mueller-Hinton agar supplemented with 5% sheep blood, incorporating compounds at concentrations from 0.002-8 μg/ml in doubling dilutions. Inocula were prepared by suspending growth from overnight cultures in Mueller-Hinton broth to a turbidity of a 0.5 McFarland standard. Final inocula contained 10⁴ organisms/spot. Plates were inoculated with a Steers replicator with 3 mm inoculating pins, and incubated overnight at 35° C. in ambient air. The lowest concentration of antibiotic showing no growth was read as the MIC. Quality control strains—Staphylococcus aureus ATCC 29913 and Streptococcus pneumoniae ATCC 49619—were included in each run.

### Example 3

### Microdilution MICs

These were determined by the method recommended by the NCCLS, (Methods for Dilution Antimicrobial Susceptibility Tests for Bacteria that Grow Aerobically, 4th Edition, NCCLS, Villanova, Pa., 1997) using cation-adjusted Mueller-Hinton broth (Difco Laboratories) supplemented with 5% lysed defibrinated horse blood. Trays were prepared in-house. Suspensions with a turbidity equivalent to that of a 0.5 McFarland standard were prepared by suspending growth from blood agar plates in 2 ml Mueller-Hinton broth. Suspensions were further diluted 1:10 to obtain a final inoculum (10 μL) containing 5×10⁵ CFU/mL. Trays were incubated 20-24 hours in ambient air at 35° C. Standard quality control strains (as above) were included in each run.

### Example 4

### E-test MICs

Standard methodology was used (Clark, et al., J. Clin. Microbiol. 36:3579-3584, 1998). Mueller-Hinton plates supplemented with 5% sheep blood (BBL Microbiology Systems, Cockeysville, Md., USA) were inoculated with a 0.5 McFarland suspension harvested from plates, and E-test strips (AB Biodisk, Solna, Sweden) placed on each. After overnight incubation at 35° C., the MIC was read as the intersect where the ellipse of growth inhibition intersects the strip. E-test MICs were performed both in air and in CO₂. E-test MICs were rounded up to the next highest doubling dilution.

### Example 5

### Disk Diffusion

This was by standard NCCLS methodology (Performance Standards for Antimicrobial Disk Susceptibility Tests, 6th Edition, NCCLS, Villanova, Pa., 1997) using 5 μg gemifloxacin disks (BBL) and Mueller-Hinton plates supplemented with 5% sheep blood (BBL), inoculated with a 0.5 McFarland suspension. After overnight incubation in both air and 5% CO₂ at 35° C., zone diameters were measured with calipers.

Each reference cited herein is hereby incorporated by reference in its entirety. Moreover, each patent application to which this application claims priority is hereby incorporated by reference in its entirety.

What is claimed is:

1. A method for modulating metabolism of ciprofloxacin-resistant and/or Streptococcus pneumoniae comprising the step of contacting ciprofloxacin-resistant Streptococcus pneumoniae with an antibacterially effective amount of a composition comprising a gemifloxacin compound, or antibacterially effective derivatives thereof.

2. The method of claim 1 wherein said ciprofloxacin-resistant Streptococcus pneumoniae is ciprofloxacin-resistant Streptococcus pneumoniae having an MIC≧8 μg/ml of ciprofloxacin.

3. A method of treating or preventing a bacterial infection by ciprofloxacin-resistant Streptococcus pneumoniae comprising the step of administering an antibacterially effective amount of a composition comprising a gemifloxacin compound to a mammal suspected of having or being at risk of having an infection with ciprofloxacin-resistant Streptococcus pneumoniae.

4. The method of claim 3 wherein said ciprofloxacin-resistant Streptococcus pneumoniae is ciprofloxacin-resistant Streptococcus pneumoniae having an MIC≧8 μg/ml of ciprofloxacin.

5. The method of claim 1 wherein said modulating metabolism is inhibiting growth of said bacteria.

6. The method of claim 1 wherein said modulating metabolism is killing said bacteria.

7. The method of claim 1 wherein said contacting said bacteria comprises the further step of introducing said composition into a mammal.

8. The method of claim 3 wherein said mammal is a human.

9. The method of claim 7 wherein said mammal is a human.

10. The method of claim 8 wherein said bacteria is selected from the group consisting of: ciprofloxacin-susceptible pneumococci having an MIC≦4 μg/ml of ciprofloxacin;
ciprofloxacin-resistant pneumococci having an MIC≧8 μg/ml of ciprofloxacin;
ciprofloxacin-susceptible Streptococcus pneumoniae having an MIC≦4 μg/ml of ciprofloxacin; and
ciprofloxacin-resistant Streptococcus pneumoniae having an MIC≧8 μg/ml of ciprofloxacin.

11. The method of claim 9 wherein said bacteria is selected from the group consisting of: ciprofloxacin-susceptible pneumococci having an MIC≦4 μg/ml of ciprofloxacin;
ciprofloxacin-resistant pneumococci having an MIC≧8 μg/ml of ciprofloxacin;
ciprofloxacin-susceptible Streptococcus pneumoniae having an MIC≦4 μg/ml of ciprofloxacin; and
ciprofloxacin-resistant Streptococcus pneumoniae having an MIC≧8 μg/ml of ciprofloxacin.

12. A method for modulating metabolism of ciprofloxacin-sensitive Streptococcus pneumoniae comprising the step of contacting ciprofloxacin-sensitive Streptococcus pneumoniae with an antibacterially effective amount of a composition comprising a gemifloxacin compound, or antibacterially effective derivatives thereof.

13. The method of claim 12 wherein said ciprofloxacin-sensitive Streptococcus pneumoniae is a ciprofloxacin-susceptible pneumococci having an MIC≦4 μg/ml of ciprofloxacin.

14. A method of treating or preventing a bacterial infection by ciprofloxacin-sensitive Streptococcus pneumoniae comprising the step of administering an antibacterially effective amount of a composition comprising a gemifloxacin compound to a mammal suspected of having or being at risk of having an infection with ciprofloxacin-sensitive Streptococcus pneumoniae.

15. The method of claim 14 wherein said ciprofloxacin-sensitive Streptococcus pneumoniae is ciprofloxacin-susceptible Streptococcus pneumoniae having an MIC≦4 μg/ml of ciprofloxacin.

16. The method of claim 12 wherein said modulating metabolism is inhibiting growth of said bacterial.

17. The method of claim 12 wherein said modulating metabolism is killing said bacterial.

18. The method of claim 12 wherein said contacting said bacterial comprises the further step of introducing said composition into a mammal.

19. The method of claim 14 wherein said mammal is a human.

20. The method of claim 18 wherein said mammal is a human.

21. The method according to claim 1, 3, 12, or 14 wherein the gemifloxacin compound is gemifloxacin or a pharmaceutically acceptable salt thereof.

22. The method according to claim 21 wherein the gemifloxacin compound is gemifloxacin mesylate or a hydrate thereof.

23. The method according to claim 22 wherein the gemifloxacin compound is gemifloxacin mesylate sesquihydrate.
